(19) United States
(12) Patent Application Publication
(10) Pub. No.: US 2025/0213545 A1
(43) Pub. Date: Jul. 3, 2025

(54) COMPOSITION FOR PREVENTION, ALLEVIATION, OR TREATMENT OF NEURODEGENERATIVE DISEASES, COMPRISING FEXOFENADINE

(71) Applicants: AJOU UNIVERSITY INDUSTRY-ACADEMIC COOPERATION FOUNDATION, Suwon-si, Gyeonggi-do (KR); STANDIGM INC., Seoul (KR)

(72) Inventors: Sang Myun PARK, Suwon-si, Gyeonggi-do (KR); Duk Yong YOON, Suwon-si, Gyeonggi-do (KR); Soojeong KIM, Seoul (KR); Tae Young HA, Suwon-si, Gyeonggi-do (KR); Jae Bong KIM, Suwon-si, Gyeonggi-do (KR); Dongkyu KIM, Seoul (KR); Yujeong KIM, Yongin-si, Gyeonggi-do (KR); Minyoung SO, Seoul (KR); Hee Jung KOO, Seoul (KR); Daehee HAN, Seoul (KR)

(21) Appl. No.: 19/100,913

(22) PCT Filed: Aug. 4, 2023
(86) PCT No.: PCT/KR2023/011489

§ 371 Date: Feb. 3, 2025

(30) Foreign Application Priority Data
Aug. 12, 2022 (KR) 10-2022-0101395

(51) Int. Cl. A61K 31/445; A61P 25/16

(52) U.S. Cl. CPC A61K 31/445; A61P 25/16

(57) ABSTRACT

The present invention relates to a composition for prevention, alleviation, or treatment of neurodegenerative diseases, comprising fexofenadine. Fexofenadine, a compound already commercialized for pharmacological purposes, has proven to be safe, demonstrated significant improvements in behavioral abilities when administered to a neurodegenerative disease model, and exhibits neuroprotective effects of inhibiting the death of dopaminergic neurons, reducing damage to inflammation-mediated neurons or neuronal degeneration, preventing the formation of α-synuclein aggregates, and suppressing the abnormal increase and activation of astrocytes, and has the effect of extending the lifespan in the degenerative neurological disease model. Thus, the composition according to the present invention can be advantageously used for preventing, alleviating, or treating neurodegenerative diseases.

### TECHNICAL FIELD

The present invention relates to a composition for preventing, alleviating or treating a neurodegenerative disease, including fexofenadine.

### BACKGROUND ART

As people age, various neurological and brain-related diseases or degenerative diseases, such as dementia, cerebrovascular disease, Parkinson's disease, depression and Alzheimer's disease, are observed at a higher frequency, and the prevalence is expected to increase further as we enter an aging society. Neurodegenerative diseases are diseases that are associated with the gradual loss of structure or function of neurons, including the death of neurons. In particular, the onset of neurodegenerative diseases such as Alzheimer's disease and Parkinson's disease is known to be closely related to the death of neurons.

In particular, Parkinson's disease is a degenerative disease of the central nervous system mainly caused by degeneration or arteriosclerotic changes in the diencephalon, and movement disorders are the main symptom. In the brain of normal people, neurons in a region called the substantia nigra are degenerated, and it is caused by a deficiency of dopamine, which a neurotransmitter produced here. Parkinson's disease is a chronic, progressive motor nervous system disease that occurs when neurons that produce dopamine are damaged, resulting in a lack of dopamine and the dominance of acetylcholine, which maintains balance. The main symptoms of this disease include tremor of the hands, arms, legs and face, stiffness of the limbs and body, bradykinesia and postural instability, which make it difficult to maintain balance.

In addition, Alzheimer's disease is the most common form of dementia in the elderly, and is characterized by pathological features such as general atrophy of the brain, enlargement of the ventricles, multiple neurofibrillary tangles and neuritic plaques, and it causes a gradual decline in intellectual functions such as memory, judgment and language ability, as well as impairments in daily living abilities, personality and behavioral patterns.

Currently, various treatments such as cell transplantation and surgical procedures have been proposed to treat neurodegenerative diseases, but most of the above have risk factors and side effects, and due to the complexity of the cell damage mechanism, no treatment has yet been developed that can actually prevent the damage or death of neurons.

Meanwhile, fexofenadine is an antihistamine medicine used to treat allergic symptoms such as seasonal allergies and chronic urticaria (Compalati, E et al, (2011). International Archives of Allergy and Immunology. 156 (1): 1-15.), and it is currently sold under the brand name Allegra. Therapeutically, fexofenadine acts as a selective peripheral H1 blocker, and is classified as a second-generation antihistamine because it does not pass through the blood-brain barrier and has less sedative effect compared to first-generation antihistamines.

In the present invention, it was confirmed through histological analysis of a Parkinson's animal model, which is an animal model of neurodegenerative disease, that fexofenadine, which has already been approved by the FDA as an antihistamine and has proven safety, can inhibit damage or death of neurons, and by confirming that fexofenadine can actually improve behavioral abilities in an animal model of neurodegenerative disease, the inventors of the present invention have demonstrated for the first time the therapeutic effect of fexofenadine on neurodegenerative diseases.

### DISCLOSURE

### Technical Tasks

An object of the present invention is to provide a novel use of fexofenadine.

### Technical Solution

In order to achieve the above object, the present invention provides a pharmaceutical composition for preventing or treating a neurodegenerative disease, including fexofenadine or a pharmaceutically acceptable salt thereof as an active ingredient.

In the pharmaceutical composition the neurodegenerative disease may be selected from the group consisting of Parkinson's disease, Alzheimer's disease, Huntington's disease, amyotrophic lateral sclerosis, multiple sclerosis, Creutzfeldt-Jakob disease, Lou Gehrig's disease, spinal amyotrophic lateral sclerosis, Niemann-Pick disease, synucleinopathy and dementia.

In the present invention, the pharmaceutical composition may further include a pharmaceutically acceptable excipient.

In the present invention, the pharmaceutical composition may protect neurons from neurotoxicity.

In the present invention, the pharmaceutical composition may protect neurons through:
(i) inhibiting dopaminergic neuron death,
(ii) inhibiting inflammation-mediated neuronal damage or neuronal degeneration;
(iii) inhibiting α-synuclein Ser-129 phosphorylation aggregate formation; and/or
(iv) inhibiting an abnormal increase in the number and/or activation of astrocytes.

In the present invention, the pharmaceutical composition may
(i) improve exercise capacity; and/or
(ii) prolong the lifespan of a subject to which the pharmaceutical composition is administered.

In addition, the present invention provides a food composition, including fexofenadine.

In the food composition, the food may be a health functional food for preventing or ameliorating a neurodegenerative disease.

In the food composition, the neurodegenerative disease may be selected from the group consisting of Parkinson's disease, Alzheimer's disease, Huntington's disease, amyotrophic lateral sclerosis, multiple sclerosis, Creutzfeldt-Jakob disease, Lou Gehrig's disease, spinal amyotrophic lateral sclerosis, Niemann-Pick disease, synucleinopathy and dementia.

In addition, the present invention provides a composition for protecting neurons, including fexofenadine as an active ingredient.

### Advantageous Effects

Fexofenadine according to the present invention is a compound already commercialized for pharmacological purposes, has proven to be safe, demonstrated significant improvements in behavioral abilities when administered to a neurodegenerative disease model, and exhibits neuroprotective effects of inhibiting the death of dopaminergic neurons, reducing damage to inflammation-mediated neurons or neuronal degeneration, preventing the formation of α-synuclein aggregates, and/or suppressing the abnormal increase and/or activation of astrocytes, and has the effect of extending the lifespan in the degenerative neurological disease. Thus, the composition according to the present invention can be advantageously used for preventing, alleviating, or treating neurodegenerative diseases.

### DESCRIPTION OF DRAWINGS

FIG. 1 shows a 6-OHDA injection site and fexofenadine administration and analysis schedule for the production of Parkinson's disease model (6-01DA model) mice.

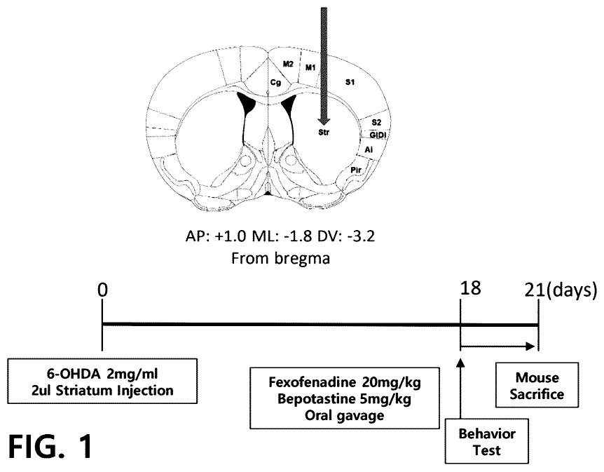

FIG. 2a shows the results of a bridge test to confirm the therapeutic effect of fexofenadine administration in Parkinson's disease model (6-OHDA model) mice (*p<0.05, **p<0.01).

FIG. 2b shows the results of a pole test to confirm the therapeutic effect of fexofenadine administration in Parkinson's disease model (6-OHDA model) mice (*p 0.5<0.05, **p<0.01, ***p<0.001).

FIG. 2c shows the results of a cylinder test to confirm the therapeutic effect of fexofenadine administration in Parkinson's disease model (6-OHDA model) mice (*p<0.05, **p<0.01, ***p<0.001).

FIG. 3 shows the results of analyzing the ratio of TH-positive neurons and the number of TH-positive neurons in the substantia nigra to confirm the therapeutic effect of fexofenadine administration in Parkinson's disease model (6-01-IDA model) mice (*p<: 0.05, **p<0.01, ***p<:0.001; and Scale bar: 50 μm).

FIG. 4 shows the results of analyzing the density of TH-positive neurons in the striatum to confirm the therapeutic effect of fexofenadine administration in Parkinson's disease model (6-OHDA model) mice (*p<0.05, **p<0.01, * **p<0.001; and Scale bar: 50 μm).

FIG. 5 shows the results of analyzing the number of activated microglia through Iba-1 staining to confirm the therapeutic effect of fexofenadine administration in Parkinson's disease model (6-OHDA model) mice (*p<0.05, ** p<0.01, ***p 0.001; and Scale bar: 50 μm).

FIG. 6 shows an injection site of α-synuclein and fexofenadine administration and analysis schedule for the production of Parkinson's disease model (α-synuclein injection model) mice.

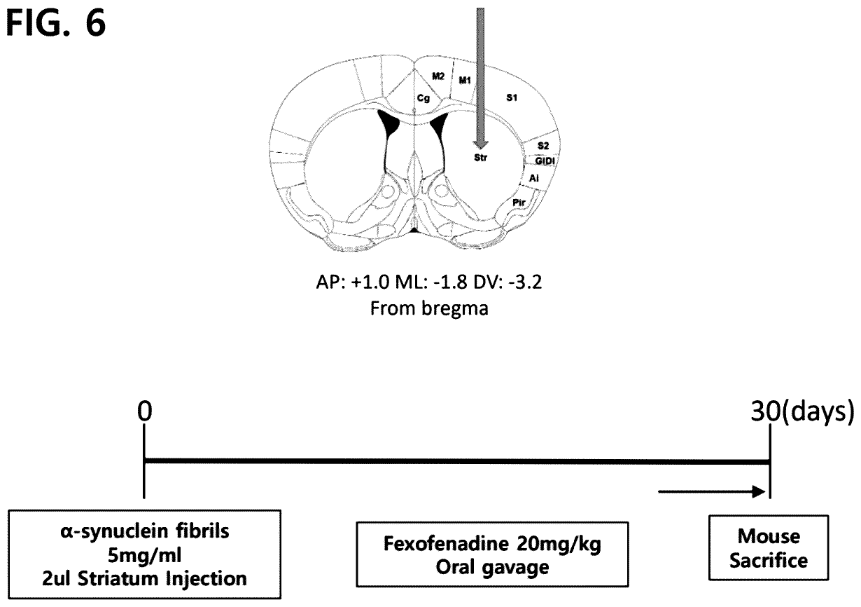

FIG. 7a shows the results of staining for α-synuclein Ser-129 phosphorylated aggregates in the cingulate cortex (Cg), primary motor cortex (M1), secondary motor cortex (M2), caudate putamen (CPu), perirhinal cortex (PRh), midbrain reticular nucleus (MRN) and substantia nigra pars compacta (SNc) on the ipsilateral and contralateral sides to which α-synuclein was injected to confirm the therapeutic effect of fexofenadine administration in Parkinson's disease model (α-synuclein injection model) mice (Scale bar: 50 μm).

FIG. 7b shows the results of quantitative analysis of the number of pSer129 α-syn pathology in various parts of the brain tissues on the ipsilateral and contralateral sides to which α-synuclein was injected to confirm the therapeutic effect of fexofenadine administration in Parkinson's disease model (α-synuclein injection model) mice (*p<0.05, **p<0.01, ***p<0.001, ****p<0.0001).

FIG. 8 shows the results of GFAP staining in the cortex, striatum and substantia nigra on the ipsilateral and contralateral sides to which α-synuclein was injected to confirm the therapeutic effect of fexofenadine administration in Parkinson's disease model (α-synuclein injection model) mice (*p<0.05, **p<,0.01, ***p<0.001; and Scale bar: 50 μm).

FIG. 9 shows the results of analyzing the number of activated microglia through Iba-1 staining in the cortex, striatum and substantia nigra on the ipsilateral and contralateral sides to which α-synuclein was injected to confirm the therapeutic effect of fexofenadine administration in Parkinson's disease model (α-synuclein injection model) mice (*p<0.05, **p<0.01, ***p<0.001; and Scale bar: 50 m).

FIG. 10a shows the results of confirming with Venus fluorescence per unit area whether fexofenadine inhibits the formation of α-synuclein aggregates in the BIFC-α-syn C. elegans Parkinson's disease model (*p<0.05).

FIG. 10b shows the results of confirming with Venus fluorescence intensity whether fexofenadine inhibits the formation of α-synuclein aggregates in the BIFC-α-syn C. elegans Parkinson's disease model (*p<0.05).

FIG. 10c shows the results of pharyngeal pumping analysis to confirm whether fexofenadine restores exercise ability in the BIFC-α-syn C. elegans Parkinson's disease model (*p<0.05).

FIG. 10d shows the results of life-span analysis to confirm the effect of fexofenadine treatment on the lifespan of the BIFC-α-syn C. elegans Parkinson's disease model (*p<0.05).

### MODES OF THE INVENTION

Unless otherwise defined, all technical and scientific terms used in the present specification have the same meanings as commonly understood by a person skilled in the art to which the present invention pertains. In general, the nomenclature used herein is well known and commonly used in the art.

In the present invention, the term “about” or “approximately” means within an acceptable error range for a specific value as determined by a person skilled in the art, and how the value is measured or determined, that is, it depends in part on the limitations of the measurement system. For example, “about” may mean within 1 or more than 1 standard deviation, depending on the practice in the art. Alternatively, “about” may mean a range of less than 20%, less than 10%, less than 5%, or less than 1% of a given value or range. Alternatively, particularly in relation to biological systems or processes, the term may mean within a magnitude of within 5 times or within 2 times a particular value. When specific values are described herein and in the claims, it should be assumed that the term “about” means within an acceptable margin of error for the specific value, unless otherwise stated.

In the present invention, the “administration” of a compound means providing a compound or a prodrug of the compound to a subject in need of treatment.

In the present invention, when fexofenadine is administered to an animal model of neurodegenerative disease, the behavioral abilities of the animal model of neurodegenerative disease are restored to the normal level, and in histological analysis, it was confirmed that it has the effects of protecting dopaminergic neurons and inhibiting inflammation-mediated neuronal damage and neuronal degeneration.

Accordingly, in one aspect, the present invention relates to a pharmaceutical composition for preventing or treating a neurodegenerative disease, including fexofenadine or a pharmaceutically acceptable salt thereof as an active ingredient.

In the present invention, “fexofenadine” may be a compound represented by Chemical Formula 1 below:

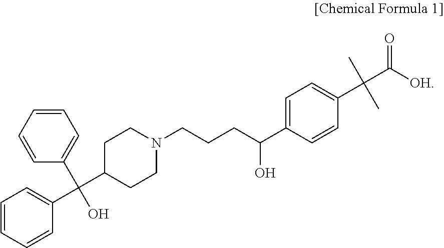 [SMILES: CC(C)(C(=O)O)c1ccc(C(O)CCCN2CCC(C(O)(c3ccccc3)c3ccccc3)CC2)cc1]

As used herein, the term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The compound of the present invention may contain several asymmetric centers and may be present in the form of optically pure enantiomers, mixtures of enantiomers such as, for example, racemates, optically pure diastereoisomers, mixtures of diastereoisomers, diastereoisomeric racemates or mixtures of diastereoisomeric racemates. According to the Cahn-Ingold-Prelog Convention, the asymmetric carbon atom may be of the “R” or “S” configuration.

In the present invention, the term “neurodegenerative disease” refers to a disease associated with symptoms caused by damage to neurons, degeneration of neurons, loss of neuron function and/or death of neurons, including Parkinson's disease and Alzheimer's disease, Huntington's disease, amyotrophic lateral sclerosis, multiple sclerosis, Creutzfeldt-Jakob disease, Lou Gehrig's disease, spinal amyotrophy, Niemann-Pick disease, synucleinopathy and dementia, but is not limited thereto.

In the present invention, the dementia may be senile dementia, diabetic dementia alcoholic dementia or vascular dementia, but is not limited thereto.

The pharmaceutical composition of the present invention may further include various excipients, including pharmaceutically acceptable diluents or carriers. In some embodiments, the pharmaceutical composition of the present invention may be provided by administering the same to a subject according to the need. In some embodiments, the pharmaceutical composition of the present invention may be administered to humans.

The pharmaceutical composition of the present invention may be administered singly or co-administered to a patient. Coadministration means involving the simultaneous or sequential administration of compounds, individually or in combination (more than one compound or agent). Accordingly, the preparation may also be combined with other active substances if desired.

The pharmaceutical composition of the present invention may be formulated and used in the form of oral dosage forms such as powders, granules, tablets, capsules, suspensions, emulsions, syrups, aerosols and the like, external preparations, suppositories or sterile injectable solutions according to conventional methods.

The pharmaceutical composition of the present invention may be administered orally or parenterally (e.g., intravenously, subcutaneously, intraperitoneally or topically) depending on the desired method.

Solid formulations for oral administration may include tablets, pills, powders, granules, capsules and the like. The solid formulations may be prepared by mixing with at least one excipient, for example, starch, calcium carbonate, sucrose, lactose, gelatin and the like. In addition to such general excipients, lubricants such as magnesium stearate or talc may also be used.

Liquid formulations for oral administration may include suspensions, solutions for internal use, emulsions, syrups and the like. In addition to simple diluents commonly used, such as water and liquid paraffin, many different excipients may also be used, for example, wetting agents, flavors, fragrances, preserves and the like.

Preparations for parenteral administration may include sterile aqueous solutions, non-aqueous solvents, suspensions, emulsions, lyophilized preparations or suppositories. The non-aqueous solutions and the suspensions may include propylene glycol, polyethylene glycol, vegetable oil such as olive oil, injectable ester such as ethyl oleate and the like.

The pharmaceutical composition may be a sterile injectable preparation as a sterile injectable aqueous or oily suspension. This suspension nay be formulated according to techniques known in the art using suitable dispersing or wetting agents (e.g. Tween 80) and suspending agents. Sterile injectable preparations may also be sterile injectable solutions or suspensions (e.g., solutions in 1,3-butanediol) in non-toxic, parenterally acceptable diluents or solvents. Vehicles and solvents that can be tolerated include mannitol, water, Ringer's solution, and isotonic sodium chloride solution. Additionally, sterile non-volatile oils may typically be used as solvents or suspending media. For this purpose, any non-irritating non-volatile oil may be used, including synthetic mono- or diglycerides. Fatty acids, such as oleic acid and its glyceride derivatives, are useful in injectable formulations, as are pharmaceutically acceptable natural oils (e.g., olive oil or castor oil), particularly, their polyoxyethylated versions.

The pharmaceutical composition of the present invention may also be administered in the form of a suppository for rectal administration. These compositions may be prepared by mixing the compound of the present invention with suitable non-irritating excipients that are solid at room temperature but liquid at rectal temperature. These substances include, but are not limited to, cocoa butter, beeswax and polyethylene glycol.

Parenteral administration of the pharmaceutical composition according to the present invention is particularly useful when the desired treatment involves areas or organs that are easily accessible by topical application. When applied topically to the skin, the pharmaceutical composition should be formulated as a suitable ointment containing the active ingredient suspended or dissolved in a carrier. Carriers for topical administration of the compounds of the present invention include, but are not limited to, mineral oil, liquid paraffin, white petroleum jelly, propylene glycol, polyoxyethylene, polyoxypropylene compounds, emulsifying wax and water. In another embodiment, the pharmaceutical composition of the present invention may be formulated as a suitable lotion or cream containing the active compound suspended or dissolved in a carrier. Suitable carriers include, but are not limited to, mineral oil, sorbitan monostearate, polysorbate 60, cetyl ester wax, cetearyl alcohol, 2-octyldodecanol, benzyl alcohol and water. The pharmaceutical composition of the invention may also be applied topically to the lower intestinal tract by rectal suppositories or with suitable enemas. Topically applied transdermal patches are also included in the present invention.

The pharmaceutical composition of the present invention may be administered by intranasal aerosol or inhalation. These compositions are prepared according to techniques well known in the art and may be prepared as solutions in saline using benzyl alcohol or other suitable preservatives, absorption promoters to enhance bioavailability, fluorocarbons and/or other solubilizing or dispersing agents known in the art.

The relative amounts of the active ingredient, pharmaceutically acceptable excipients and/or any additional ingredients in the pharmaceutical composition of the invention will vary depending on the identity, size and/or disorder of the subject being treated and the route by which the composition is administered. For example, the content of the active ingredient included in the pharmaceutical composition of the present invention is not particularly limited, but it may be included in an amount of 0.0001 to 100 wt %, for example, 0.001 to 50 wt %, and more preferably, 0.01 to 10 wt %, based on the total weight of the final composition.

In the present invention, the pharmaceutically acceptable excipients include all solvents, dispersion media, diluents, or other liquid vehicles, dispersions, suspension aids, surfactants, isotonic agents, thickeners, or emulsifiers, preservatives, solid binders and lubricants, which are suitable for a particular dosage form. Remington's literature [The Science and Practice of Pharmacy, 21st Edition, A. R. Gennaro, (Lippincott, Williams & Wilkins, Baltimore, MD, 2006] discloses various excipients used in the preparation of pharmaceutical compositions and known techniques for their preparation. Any conventional carrier medium is considered to be within the scope of the present invention, except when it is incompatible with the substance or its derivatives by providing any undesirable biological effects or otherwise interacting in a deleterious manner with any other components of the pharmaceutical composition. Pharmaceutically acceptable excipients are at least 95%, 96%, 97%, 98%, 99% or 100% pure.

The excipients are approved for human and veterinary use. In some embodiments, the excipients are approved by the US FDA, In some embodiments, the excipients are pharmaceutical grade. In some embodiments, the excipients meet the standards of the United States Pharmacopoeia (USP), European Pharmacopoeia (EP), British Pharmacopoeia, and/or International Pharmacopoeia (EP)

The pharmaceutically acceptable excipients used in the preparation of the pharmaceutical composition may include inert diluents, dispersants and/or granulating agents, surfactants and/or emulsifiers, disintegrants, binders, preservatives, buffers, lubricants, and/or oils, but the present invention is not limited thereto.

Exemplary diluents include calcium carbonate, sodium carbonate, calcium phosphate, dicalcium phosphate, calcium sulfate, calcium hydrogen phosphate, sodium lactose phosphate, sucrose, cellulose, microcrystalline cellulose, kaolin, mannitol, sorbitol, inositol, sodium chloride, dry, starch, corn starch, powdered sugar and a combination thereof, but the present invention is not limited thereto.

Exemplary granulating agents and/or dispersing agents include potato starch, corn starch, tapioca starch, sodium starch glycolate, clay, alginic acid, guar gum, citrus pulp, agar, bentonite, cellulose and wood products, natural sponges, cation-exchange resins, calcium carbonate, silicate, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (Starch 1500), microcrystalline starch, water-insoluble starch, calcium carboxymethyl cellulose, magnesium aluminum silicate (Veegum), sodium lauryl sulfate, quatemary ammonium compounds, and a combination thereof, but the present invention is not limited thereto.

Exemplary surfactants and/or emulsifiers include natural emulsifiers (e.g., acacia, agar, alginic acid, sodium alginate, tragacanth, chondrux, cholesterol, xanthan, pectin, gelatin, egg yolk, casein, wool fat, wax, and lecithin), colloidal clays (e.g., bentonite [aluminum silicate] and Veegum [magnesium aluminum silicate]), long-chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, and polyvinyl alcohol), carbomers (e.g., carboxy polymethylene, polyacrylic acid, acrylic acid polymers, and carboxyvinyl polymers), carrageenan, cellulose derivatives (e.g., carboxymethyl cellulose sodium, powdered cellulose, hydroxymethyl cellulose, hydroxypropyl cellulose, hydroxypropyl methylcellulose, and methyl cellulose), sorbitan fatty acid esters (e.g., polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g., polyoxyethylene monostearate [Myrj 45], polyoxyethylene hydrogenated castor oil, poly ethoxylated castor oil, polyoxymethylene stearate, and Solutol), sucrose fatty acid esters, polyethylene glycol fatty acid esters (e.g., Cremophor), polyoxyethylene ethers (e.g., polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetyl pyridinium chloride, benzalkonium chloride, docusate sodium, and/or a combination thereof, but the present invention is not limited thereto.

Exemplary binders include starches (e.g. con starch and starch paste): gelatin; sugars (e.g. sucrose, glucose, dextrose, dextrin, molasses, lactose, lactitol, and mannitol); natural and synthetic gums (e.g. acacia, sodium alginate, Irish moss extract, panwar gum, ghatti gum, mucilage of isapgol husks, carboxymethylcellulose, methylcellulose, ethylcellulose, hydroxyethylcellulose, hydroxypropylcellulose, hydroxypropyl methylcellulose, microcrystalline cellulose, cellulose acetate, poly(vinyl-pyrrolidone), magnesium aluminum silicate (Veegum), and larch arabinogalactan); alginates; polyethylene oxides; polyethylene glycols: inorganic calcium salts; silicic acid; polymethacrylates; waxes; water: alcohols; and a combination thereof, but the present invention is not limited thereto.

Exemplary preservatives may include antioxidants, chelating agents, antimicrobial preservatives, antifungal preservatives, alcohol preservatives, acidic preservatives, and other preservatives. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite, but the present invention is not limited thereto. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal, but the present invention is not limited thereto. Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid, but the present invention is not limited thereto. Exemplary alcohol preservatives include ethanol, polyethylene glycol, phenol, phenol-based compounds, bisphenol, chlorobutanol, hydroxybenzoate, and phenylethyl alcohol, but the present invention is not limited thereto. Exemplary acidic preservatives include vitamin A, vitamin C, vitamin E, beta-carotene, citric acid, acetic acid, dehydroacetic acid, ascorbic acid, sorbic acid, and phytic acid, but the present invention is not limited thereto. Other preservatives include tocopherol, tocopherol acetate, deteroxime mesylate, cetrimide, butylated hydroxyanisole (BTA), butylated hydroxytoluende (BI-IT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methyl paraben, Germall 115, Germaben II, Neolone, Kathon, and Euxyl, but the present invention is not limited thereto. In a specific embodiment, the preservative is an antioxidant. In another embodiment, the preservative is a chelating agent.

Exemplary buffers include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and a combination thereof, but the present invention is not limited thereto.

Exemplary lubricants include magnesium stearate, calcium stearate, stearic acid, silica, tale, malt, glyceryl behenate, hydrogenated vegetable oils, polyethylene glycol, sodium benzoate, sodium acetate, sodium chloride, leucine, magnesium lauryl sulfate, sodium lauryl sulfate, and a combination thereof, but the present invention is not limited thereto.

Exemplary oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savory, sea buckthorn, sesame, shea butter, silicone, soybean, sunflower, tea tree, thistle, tsubaki, vetiver, walnut, and wheat germ oils, but the present invention is not limited thereto. Exemplary oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and a combination thereof, but the present invention is not limited thereto.

Such excipients may optionally be included in the preparation of the present invention. Excipients such as cocoa butter and suppository waxes, colorants, coating agents, sweeteners, flavoring agents, and perfumes may be present in the composition at the discretion of the formulator.

As used herein, the term “prevention” includes preventing or delaying the appearance of clinical or subclinical symptoms of a condition, disorder or disease in a mammal, particularly in an individual that may be susceptible to or has the condition, disorder or disease, but has not yet experienced or exhibited clinical or subclinical symptoms of the condition, disorder or disease.

The term “treatment” of the present invention includes
(1) inhibiting a condition, disorder or disease (e.g., inhibiting, reducing or delaying the onset of a disease, or in the case of maintenance treatment, recurrence of at least one of the clinical or subclinical symptoms); and/or
(2) alleviating a condition, disorder or disease (i.e., causing regression of the condition, disorder or disease, or at least one of its clinical or subclinical symptoms).

The benefit to the patient receiving treatment may be statistically significant, or at least perceptible to the patient or physician. However, those skilled in the art will understand that when drugs are administered to a patient to treat a disease, the result may not always be an effective treatment.

Although the pharmaceutical composition provided in the present invention is principally directed to a pharmaceutical composition for administration to humans, those skilled in the art will appreciate that such a composition is generally suitable for administration to all types of animals, That is, the pharmaceutical composition according to the present invention may also be administered to other mammals, such as animals in need of veterinary treatment, such as livestock (e.g., dogs, cats, etc.), farm animals (e.g., cows, sheep, pigs, horses, etc.) and laboratory animals (e.g., rats, mice, guinea pigs, etc.). A skilled veterinary pharmacologist, who is well aware of the modifications of pharmaceutical compositions for administration to various animals, may design and/or perform such modifications simply by routine experimentation, if necessary.

The pharmaceutical composition described herein may be prepared by any method known in the field of pharmacology or as discussed below in the text. Generally, such a preparation method includes mixing the active ingredient with an excipient and/or one or more other auxiliary ingredients, and if needed or desired, forming and/or packaging the resulting product into desired single- or multi-dose units.

The pharmaceutical composition of the present invention may be manufactured, packaged and/or sold unpackaged in a single unit dose and/or a plurality of single unit doses. As used in the present invention, the term “unit dose” is an individual amount of a pharmaceutical composition including a predetermined amount of active ingredient. The amount of active ingredient is generally equal to the dose of active ingredient administered to a subject and/or a convenient fraction of such a dose, for example, ½ or ⅓ of the dose.

In the present invention, the term “administration” means introducing a predetermined substance into a patient by any appropriate method, and the administration route of the pharmaceutical composition may be administered through any general route as long as the drug can reach the target tissue. The route of administration may include, but is not limited to, intraperitoneal administration, intravenous administration, intramuscular administration, subcutaneous administration, intradermal administration, oral administration, topical administration, intranasal administration, intrapulmonary administration and intrarectal administration.

The pharmaceutical composition of the present invention may be administered in a pharmaceutically effective amount.

The term “pharmaceutically effective amount” of the present invention may mean a “therapeutically-effective amount”, which refers to an amount of a compound or composition (e.g., a compound or composition of the present invention) that is sufficient to achieve a beneficial or desired result. The pharmaceutically effective amount may be administered in one or more administrations, applications or dosages and is not intended to be limited to any particular formulation or route of administration.

The effective dosage level may be determined depending on factors including the severity of the disease, the activity of the drug, the age, weight, health, gender of the patient, the patients sensitivity to the drug, the time of administration of the composition of the present invention used, the route of administration and the excretion rate, the duration of treatment, drugs used in combination or concomitantly with the composition of the present invention used, and other factors well known in the medical field.

The pharmaceutical composition of the present invention may be administered as an individual therapeutic agent or in combination with other therapeutic agents, and may be administered sequentially or simultaneously with conventional therapeutic agents. In addition, it may be administered single or multiple times. It is important to consider all of the above factors and administer the amount that will achieve the maximum effect with the minimum amount without side effects.

The dosage of the pharmaceutical composition of the present invention may be determined by a person skilled in the art by considering the purpose of use, the degree of addiction of the disease, the patient's age, weight, gender, antecedent history or the type of substance used as an active ingredient. For reference, Allegra is administered orally in tablet form, 120 mg once a day with water.

In the present invention, the pharmaceutical composition according to the present invention including fexofenadine as an active ingredient may be characterized as protecting neurons from neuron toxicity.

For example, the pharmaceutical composition may protect neurons through
(i) inhibiting dopaminergic neuron death;
(ii) inhibiting inflammation-mediated neuronal damage or neuronal degeneration;
(iii) inhibiting α-synuclein Ser-129 phosphorylation aggregate formation; and/or
(iv) inhibiting an abnormal increase in the number and/or activation of astrocytes.

For example, in the present invention, fexofenadine may be characterized by inhibiting the activation of microglial cells, thereby inhibiting inflammation-mediated neuronal damage and neuronal degeneration, and exerting an effect of protecting neurons.

Additionally, in the present invention, the pharmaceutical composition including fexofenadine as an active ingredient may (i) improve exercise ability; and/or (ii) extending the lifespan of a subject to which the pharmaceutical composition is administered, but the present invention is not limited thereto.

From another aspect, the present invention relates to a method for preventing or treating a neurodegenerative disease, including administering fexofenadine to a subject in need thereof.

In still another aspect, the present invention relates to the use of fexofenadine in the prevention or treatment of a neurodegenerative disease.

In still another aspect, the present invention relates to the use of fexofenadine in the manufacture of a drug for preventing or treating a neurodegenerative disease.

The term “subject” of the present invention refers to all animals, including humans, which have already developed or are likely to develop a neurodegenerative disease, and the disease can be effectively prevented and/or treated by administering the composition of the present invention to the subject.

In still another aspect, the present invention relates to a food composition, including fexofenadine.

In a preferred embodiment, the food may be a health functional food for preventing or ameliorating a neurodegenerative disease.

As used herein, the term “amelioration” refers to any action that reduces at least the severity of a parameter related to the condition being treated, such as a symptom.

In the food composition according to the present invention, the term “neurodegenerative disease” means a disease associated with symptoms caused by neuronal damage, neuronal degeneration, neuron function loss and/or neuron death, and it may be characterized by being selected from the group consisting of Parkinson's disease, Alzheimer's disease, Huntington's disease, amyotrophic lateral sclerosis, multiple sclerosis, Creutzfeldt-Jakob disease, Lou Gehrig's disease, spinal amyotrophic lateral sclerosis, Niemann-Pick disease, synucleinopathy and dementia, but the present invention is not limited thereto.

In the food composition according to the present invention, the dementia may be senile dementia, diabetic dementia, alcoholic dementia or vascular dementia, but is not limited thereto.

When using the food composition of the present invention as a food additive, the composition may be added as is or used together with other foods or food ingredients, and may be used appropriately according to conventional methods. In general, when producing food or beverages, the composition of the present invention may be added in an amount of 15% by weight or less, preferably 10% by weight or less, based on the raw materials. However, in the case of long-term intake for health purposes or health control, it may be added in an amount of 5% by weight or less, and since there is no problem in terms of safety, the active ingredient may be added in amounts exceeding the above range.

The food composition of the present invention may be manufactured in all forms such as food additives, nutritional supplements and health functional foods.

For example, as a health functional food, the composition of the present invention may be prepared and consumed in the form of tea, juice and drink, or may be consumed by granulating, encapsulating and powdering. In addition, the composition of the present invention may be added to and manufactured into health drinks (including alcoholic beverages), fruits and processed foods thereof (e.g., canned fruits, bottled fruits, jams, marmalades, etc.), fish, meats and processed foods thereof (e.g., ham, sausages, corned beef, etc.), breads and noodles (e.g., udon, buckwheat noodles, ramen, spaghetti, macaroni, etc.), fruit juices, various drinks, cookies, taffy, dairy products (e.g., butter, cheese, etc.), edible vegetable oils, margarine, vegetable proteins, retort foods, frozen foods, various seasonings (e.g., soybean paste, soy sauce, sauces, etc.) and the like.

When fexofenadine is manufactured by being contained in a health drink, it may contain various flavoring agents or natural carbohydrates as additional ingredients, like regular drinks. For the above-mentioned natural carbohydrate, monosaccharides such as glucose and fructose, disaccharides such as maltose and sucrose, natural sweeteners such as dextrin and cyclodextrin and synthetic sweeteners such as saccharin and aspartame may be used. The ratio of the natural carbohydrates may be appropriately determined by the selection of a person skilled in the art.

In addition to the above, the food composition of the present invention may contain various nutrients, vitamins, electrolytes, flavoring agents, coloring agents, pectic acid and its salts, alginic acid and its salts, organic acids, protective colloid thickeners, pH regulators, stabilizers, preservatives, glycerin, alcohol, carbonating agents used in carbonated beverages and the like. In addition, the food composition of the present invention may contain pulp for the production of natural fruit juice, fruit juice drinks and vegetable drinks. These ingredients may be used independently or in combination. The proportions of these additives may also be appropriately selected by those skilled in the art.

From still another aspect, the present invention relates to a composition for protecting neurons, including fexofenadine as an active ingredient.

The prevention, treatment, and alleviation effects of fexofenadine in the compositions of various embodiments according to the present invention may be equally applied to neurodegenerative diseases caused by various causes.

In the present invention, the terms “individual”, “patient” and “subject” are used interchangeably and include any animal, including mammals, such as mice, rats, other rodents, rabbits, dogs, cats, pigs, cows, sheep, horses or primates, including humans.

### EXAMPLE

Hereinafter, the present invention will be described in more detail through examples. These examples are only for illustrating the present invention, and it will be apparent to those skilled in the art that the scope of the present invention is not to be construed as limited by these examples.

### Example 1. Production of Parkinson's Disease Animal Model (6-OHDA Model) and Drug Administration

Oxydopamine, which is also known as 6-hydroxydopamine (6-OHDA) or 2,4,5-trihydroxyphenethylamine, is a neurotoxic synthetic organic compound that researchers use to selectively destroy dopaminergic and noradrenergic neurons in the brain, and is used to develop and test new drugs and treatments for Parkinson's disease, and is known to induce Parkinson's disease in laboratory animals by damaging dopaminergic neurons in the substantia nigra (Simola, Nicola et al. (2007). “The 6-Hydroxydopamine model of Parkinson's disease”. Neurotoxicity Research. 11 (3): 151-167. Reference), Accordingly, a Parkinson's disease animal model was produced by injecting 6-OHDA.

Specifically, 8-week-old C57BL/6 mice (Daehan Biolink, Eumseong, North Chungcheong Province, Korea) were anesthetized by intraperitoneal (i.p.) injection of 200 mg/kg of 2,2,2-tribromoethanol (Sigma-Aldrich), and they were placed in a stereotaxic apparatus (David Kopf Instruments, Tujunga, CA, USA) equipped With a mouse adapter. Using a Hamilton syringe (Hamilton, Bonaduz, Switzerland), 2 μL of 6-OHDA-HCl solution (2 mg/mL) (Sigma Aldrich) in 0.02% ascorbic acid was injected into the right striatum at the following stereotactic coordinates (mm from the bregma): IP+1.0; ML-1.8; DV −3.2. The skin was sutured, and the animal was removed from the stereotaxic apparatus and placed on a heating pad for 30 minutes. Normal control mice for the diseased mouse model were injected with a vehicle (0.02% ascorbic acid) instead of 6-OHDA-HCl solution.

Thereafter, after a 3-day recovery period after surgery on the disease model or normal control mice, 20 mg/kg of PBS (drug control) or fexofenadine (Sigma Aldrich) dissolved in PBS was orally administered once a day, and the experiment was performed. Behavioral experiments were performed 18 days after 6-01-IDA injection, and mice were sacrificed 21 days after 6-01-IDA injection, and histological analysis was performed.

The experiment was divided into the following 4 groups:
Normal mouse control group (PBS-Control): Vehicle injection followed by PBS administration (n=13),
Normal mouse drug administration group (PBS-Fexofenadine): Vehicle injection followed by fexofenadine administration (n=−5),
Diseased mouse control group (6-OHDA): 6-OHDA injection followed by 0.0 PBS administration (n=12), and
Diseased mouse drug administration group (6-OHDA+fexofenadine): 6-OHDA injection followed by fexofenadine administration (n=13)

### Example 2. Behavioral Test in Parkinson's Disease Animal Model (6-OHDA Model)

### 2-1. Bridge Test

In order to test the animal model's sense of balance, a bridge test was performed to measure the time it took to pass from one side to the other side on a 1 m long bar (acrossing time).

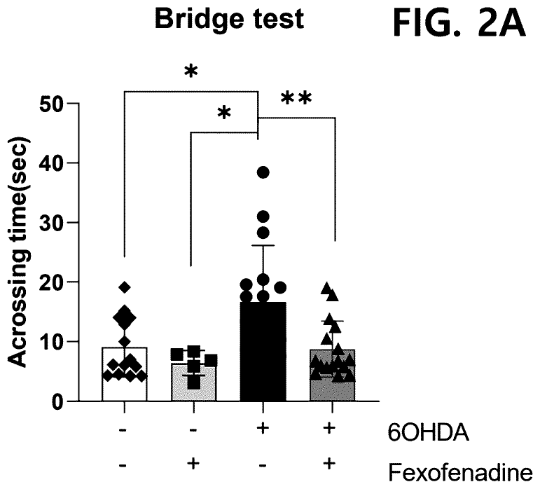

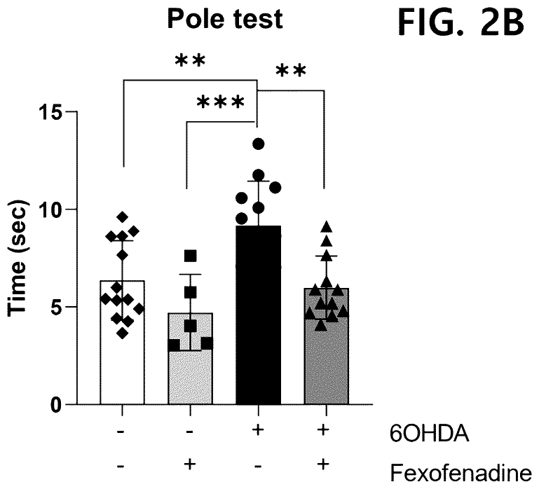

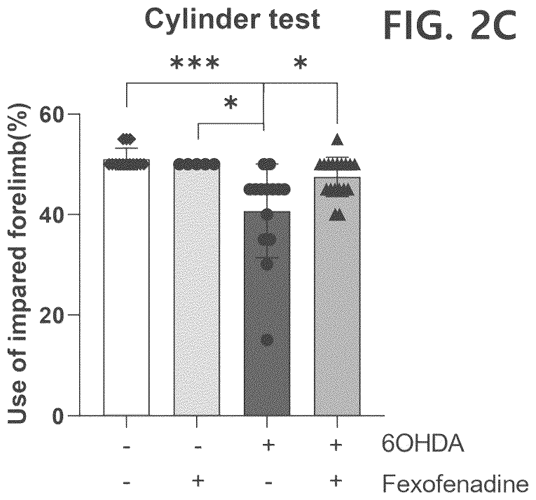

As a result, as shown in FIG. 2a, the acrossing time in the diseased mouse control group (6-OHDA) increased by about 2 times compared to the normal mouse control group (PBS-Control), but in the diseased mouse drug administration group (6-OHDA+fexofenadine) to which the fexofenadine drug was administered in the Parkinson's animal model, the acrossing time was restored to a level similar to that of the normal mouse control group (PBS-Control).

Meanwhile, it was confirmed that the acrossing time of the normal mouse drug administration group (PBS-fexofenadine) in which fexofenadine was administered to normal mice was improved compared to the normal mouse control group (PBS-Control), and it was found that it has the effect of improving the sense of balance even in normal subjects.

### 2-2. Pole Test

The mouse was placed on a bar with a diameter of 8 mm and a length of 55 cm, and a pole test was performed to measure the time it took for the mouse's four feet to come down to the floor (landing time).

As a result, as shown in FIG. 2b, the landing time of the mouse in the diseased mouse control group (6-OHDA) was about 8 to 9 seconds, and the landing ability was significantly reduced compared to the landing time of about 6 seconds in the normal mouse control group (PBS-Control), and in the diseased mouse drug administration group (6-OHDA+fexofenadine), the landing time was about 6 seconds, which confirmed that administration of fexofenadine drug restored the landing ability of Parkinson's disease model mice to the normal level.

Meanwhile, the landing time of the normal mouse drug administration group (PBS-fexofenadine) was approximately 4.5 seconds, and it was confirmed that the landing ability was improved compared to the normal mouse control group (PBS-Control), and thus, it was confirmed again that fexofenadine has the effect of improving behavioral ability even in normal subjects.

### 3. Cylinder Test

A cylinder test was performed in which a mouse was placed in a beaker with a diameter of 20 cm and a height of 40 cm, and the number of times the mouse raised its body and touched the beaker wall with its front paws was recorded. The results were expressed as a percentage of the number of steps on the forepaw opposite the damaged striatum compared to the forepaw opposite the undamaged striatum.

As a result, as shown in FIG. 2c, compared to the normal mouse control group (PBS-Control), the number of steps taken by the contralateral forelimb of the damaged stratum was reduced by nearly 10% in the diseased mouse control group (6-OHDA) compared to the contralateral forelimb of the undamaged striatum. However, in the diseased mouse drug administration group (6-OHDA+fexofenadine), it was confirmed that motor ability was restored to the level of the normal mouse control group (PBS-Control).

### Example 3. Histological Analysis in Parkinson's Disease Animal Model (6-OHDA Model)

### 3-1. TH (Tyrosine Hydroxylase) Staining Results in Substantia Nigra and Striatum

Tyrosine hydroxylase (also called tyrosine 3-monooxygenase) is an enzyme that catalyzes the conversion of the amino acid L-tyrosine to L-3,4-dihydroxyphenylalanine (L-DOPA), and it is known as a marker for dopamine, norepinephrine and epinephrine-containing (catecholamine) neurons and endocrine cells. In order to determine whether dopaminergic neurons were protected by fexofenadine administration in the Parkinson's disease animal model, an experiment was conducted to stain TH-positive cells.

Specifically, the mouse in Example 1 was anesthetized with urethane, the chest was cut, perfusion was performed by flowing a perfusate through the left ventricle of the heart for 2 minutes, and the mouse brain was separated. The mouse brain was fixed by soaking in 4% paraformaldehyde, transferred to 30% sucrose, dehydrated, cut into 35 pin thick coronal sections on a freezing microtome, and incubated with cryoprotectants (25%+ethylene glycol, 25% glycerol, and 0.05M phosphate buffer) and stored. Tissue staining was performed using 6 sheets per mouse in the area including the striatum (from 0.98 mm to −0.10 mm from the vertex) and the area including the substantia nigra (from −2.80 mm to −3.88 mm from the vertex).

Before immunostaining, brain tissue sections were washed three times with PBS in 24 wells and then treated with 3% hydrogen peroxide for 5 minutes to remove endogenous peroxidase activity. Afterwards, for dopaminergic neuron staining, primary rabbit anti-TH antibody (Abcam, ab137721, 1:1,000 dilution) was added to PBS including 0.2% Triton X-100 and 1% BSA and incubated overnight at 4° C. Afterwards, the cells were incubated with a secondary antibody (biotinylated anti-rabbit IgG, diluted 1:1,000) at 4° C. for 1 hour, and then, ABC solution was added at room temperature and incubated for additional 1 hour. Tissue sections were colored with DAB for 2 minutes, attached to gelatin-coated slides, dehydrated by increasing the ethanol concentration starting from 70% ethanol to 80, 90 and 100%, and reacted with xylene for one day, followed by mounting.

Images were taken using an optical microscope (Olympus Microscope System BX51; Tokyo, Japan) and quantified using Image J software (Bethesda, MD, USA). Optical density measurements or cell number measurements were used as the average value of the values of six sections of tissue per mouse, and they were expressed as the ratio of the density or number of TH-positive neurons on the right ipsilateral side of the brain that was damaged compared to the TH-positive neurons on the contralateral side that w as not damaged by 6-OHDA.

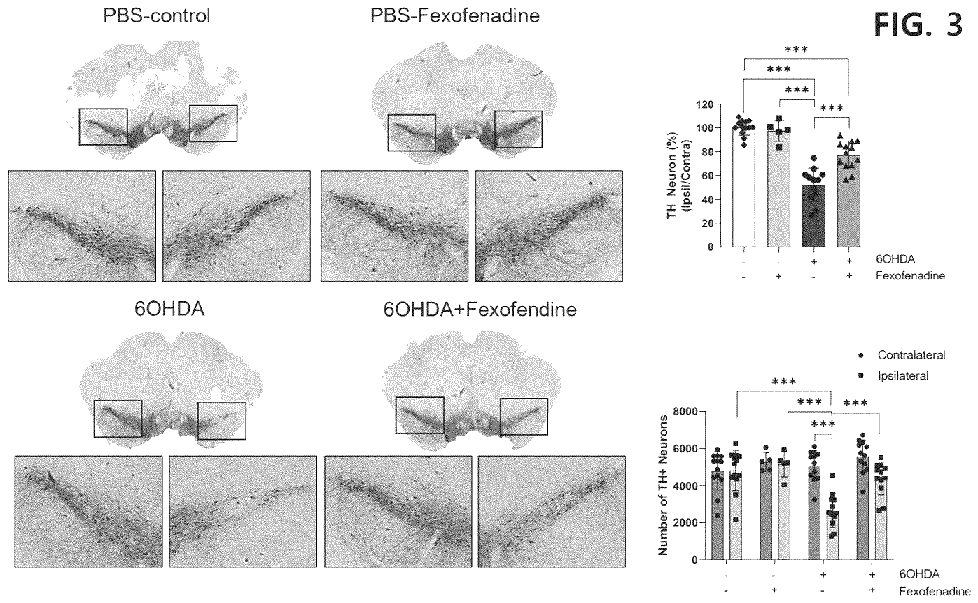

As a result, as shown in FIG. 3, in the diseased mouse control group (6-OHDA), the ratio of TH-positive neurons on the ipsilateral side compared to the contralateral side of the substantia nigra, was reduced by about 50%, but in the diseased mouse drug administration group (6-OHDA+fexofenadine), it was confirmed that the ratio of TH-positive neurons on the ipsilateral side of the substantia nigra was restored to nearly 80% compared to the contralateral side.

In addition, whereas the diseased mouse control group (6-OHDA) had a significantly reduced number of TH-positive neurons on the ipsilateral side of the substantia nigra compared to the normal mouse control group (PBS-Control), it was confirmed that in the diseased mouse drug administration group (6-OHDA+fexofenadine), the number of TH-positive neurons recovered to a level similar to that of the normal mouse control group (PBS-Control).

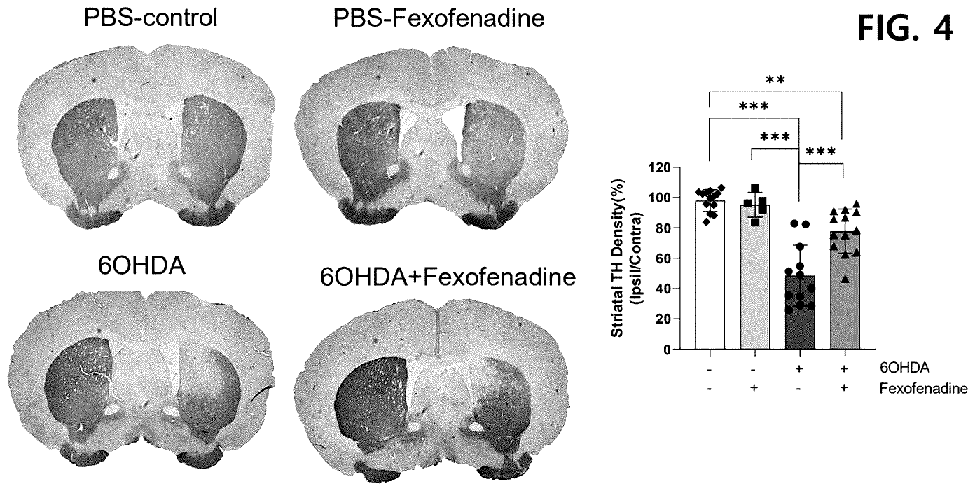

Meanwhile, as shown in FIG. 4, a similar pattern was confirmed in the striatum, and in the diseased mouse control group (6-OHDA), the density of TH-positive neurons on the ipsilateral side compared to the contralateral side was reduced to about 50% compared to the normal mouse control group (PBS-Control), whereas it was confirmed that in the diseased mouse drug administration group (6-OHDA+fexofenadine), the density of TH-positive neurons on the ipsilateral side compared to the contralateral side was recovered by nearly 80% compared to the normal mouse control group (PBS-Control).

From the above results, it was found that fexofenadine has the effect of suppressing the death of dopaminergic neurons or protecting dopaminergic neurons against neuronal cell toxicity.

### 3-2. Microglial Staining Results in Substantia Nigra and Striatum

Microglia display a surveillance mode in a normal state, but as they become activated, they sequentially go through inflammatory and anti-inflammatory phases, and are known to induce inflammation-mediated damage to neurons and degeneration of neurons. Accordingly, the effect of fexofenadine on microglial activation was analyzed.

Activated microglia were analyzed through staining with anti-Iba-1 antibody (Wako, 019-19741, 1:1,000 dilution). The brain tissue preparation and tissue staining method for this purpose were performed in the same manner as the TH staining method in Example 3-1, and the images were analyzed using an Axio scan. Z1 slide scanner (Carl Zeiss, Germany). The cell number was measured and analyzed as the average value of three sheets of each tissue.

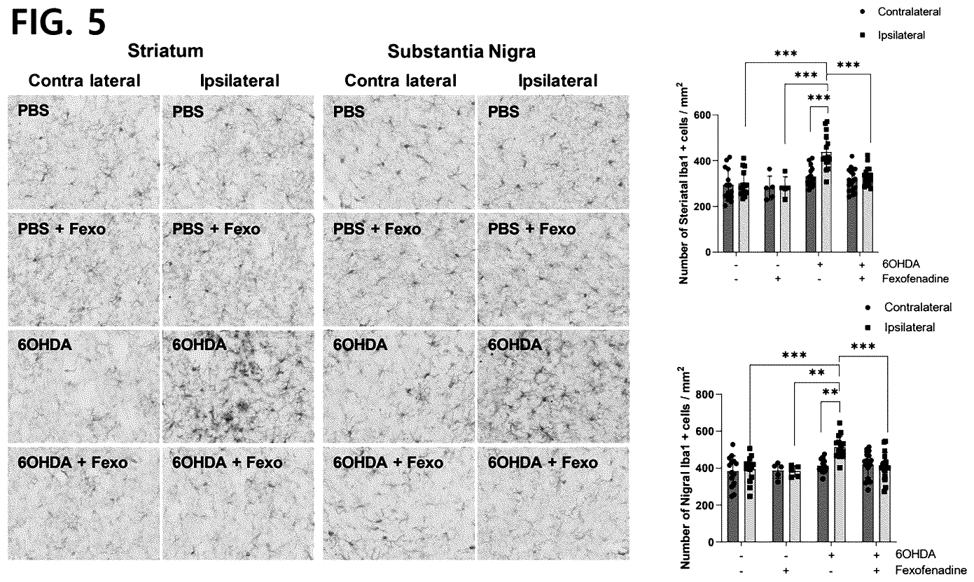

As a result, as shown in FIG. 5, the number of Iba-1 positive microglia was increased in the ipsilateral side of the striatum and substantia nigra in the diseased mouse control group (6-OHDA) compared to the normal mouse control group (PBS-Control), whereas it was confirmed that in the diseased mouse drug administration group (6-OHDA+fexofenadine), the number of Iba-1 positive microglia was reduced to a level similar to that of the normal mouse control group (PBS-Control).

From the above results, it was confirmed that fexofenadine has the effect of suppressing inflammation-mediated damage and degeneration of neurons and protecting neurons by suppressing the activation of microglial cells against neuron toxicity.

### Example 4. Production of Parkinson's Disease Animal Model (k-Synuclein Injection Model) and Drug Administration

It is known that injecting α-synuclein prepared as a recombinant protein into the striatum of mice induces Parkinson's disease by recruiting endogenous α-synuclein in mice (Lot et al. 2012, Science. 2012 Nov. 16; 338(6109)):949-53). Therefore, a Parkinson's disease animal model was produced by injecting α-synuclein, by referring to the method described in the above literature.

Specifically, 8-week-old C57BL/6 mice (Daeban Biolink, Eumseong, North Chungcheong Province, Korea) were anesthetized by intraperitoneal (i.p.) injection of 200 mg/kg of 22,2-tribromoethanol (Sigma-Aldrich), and they were placed in a stereotaxic apparatus (David Kopf Instruments, Tujunga, CA, USA) equipped with a mouse adapter. Using a Hamilton syringe (Hamilton, Bonaduz, Switzerland), 2 μL. of α-synuclein, which was prepared as a recombinant protein purified from BL21 Escherichia coli strain transformed with 5 mg/mL pRK172 α-synuclein plasmid, was injected into the following stereotaxic coordinates (mm from the bregma) in the right striatum: AP+1.0; ML-1.8; DV−3.2. The skin was sutured, and the animal was removed from the stereotaxic apparatus and placed on a heating pad for 30 minutes.

Thereafter, after a 3-day recovery period after surgery, the diseased model mice were orally administered with 20 mg/kg of fexofenadine (Sigma Aldrich) dissolved in PBS (drug control) or PBS once a day, and the experiment was performed. Mice were sacrificed on day 30, and histological analysis was performed. Control mice were injected with a vehicle (PBS) instead of α-synuclein.

The experiment was divided into three groups:
Normal mouse control group (PBS): Vehicle injection followed by PBS administration (n=3);
Diseased mouse control group (PFF-PBS): α-synuclein injection followed by PBS administration (n=6), and
Diseased mouse drug administration group (PFF-fexofenadine): α-synuclein injection followed by fexofenadine administration (n=7)

### Example 5. Histological Analysis in Parkinson's Disease Animal Model (α-Synuclein Injection Model)

### 5-1. α-Synuclein Ser-129 Phosphorylated Aggregate Staining Results

It is known that when α-synuclein is injected into the striatum of a mouse, it recruits endogenous α-synuclein of the mouse and forms α-synuclein Ser-129 phosphorylated aggregates inside neurons in the cortex, amygdala and substantia nigra, thereby causing neuronal death and damaging nerve functions.

Accordingly, the inventors of the present invention attempted to determine whether fexofenadine could inhibit the formation of α-synuclein Ser-129 phosphorylated aggregates using the Parkinson's disease animal model prepared in Example 4.

For this purpose, tissue staining was performed in the cingulate cortex (Cg), primary motor cortex (M1), secondary motor cortex (M2), caudate putamen (CPu), perirhinal cortex (PRh), midbrain reticular nucleus (MRN), and substantia nigra pars compacta (SNc) on the ipsilateral and contralateral sides to which α-synuclein was injected using pSeri29 α-syn antibody (Wako, 1:5,000 dilution) in the same manner as described in Example 3-2.

In addition, brain tissue was separated from various locations on the ipsilateral and contralateral sides to which α-synuclein was injected, and the number of pSer129 α-syn pathology was quantitatively analyzed using Zenblue 3.1 software.

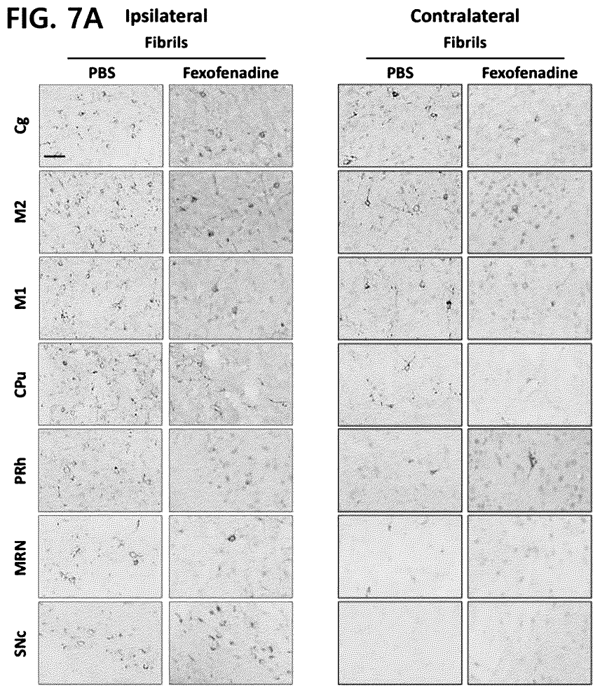

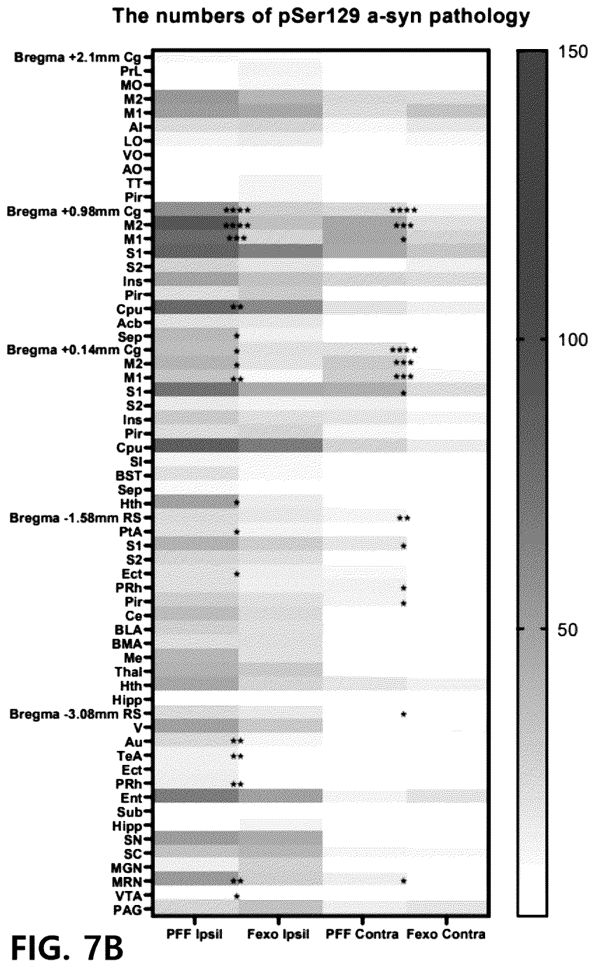

As a result, as shown in FIGS. 7a and 7b, in the diseased mouse control group (PFF-PBS), α-synuclein Ser-129 phosphorylated aggregates were generally observed at very high levels in various brain tissues on the ipsilateral and contralateral sides to which α-synuclein was injected. However, in the diseased mouse drug administration group (PFF-fexofenadine), it was confirmed that the formation of α-synuclein Ser-129 phosphorylated aggregates was inhibited, and this aspect was observed more prominently on the ipsilateral side to which α-synuclein was injected.

From the above results, it was found that fexofenadine can inhibit the death of neurons and damage to nerve functions by inhibiting the formation of α-synuclein Ser-129 phosphorylated aggregates.

### 5-2. GFAP Staining Results in Cortex, Striatum and Substantia Nigra

Glial fibrillary acidic protein (GFAP) is an intermediate microfilament protein of astrocytes, and the expression of GFAP is regulated by various stages of post-traumatic signals and nerve activity. In particular, GFAP is used as a marker to show an abnormal increase in the number or activation of astrocytes (astrogliosis) due to the destruction of nerve cells caused by damage or stress in the central nervous system (CNS).

Accordingly, using the Parkinson's disease animal model produced in Example 4, the inventors of the present invention attempted to determine whether fexofenadine could inhibit an abnormal increase or activation of the number of astrocytes.

For this purpose, 6 sections per mouse were used from the area including the cortex (0.98 mm to −0.10 mim from the bregma), the area including the striatum (0.98 mm to −0.10 mm from the bregma), and the area including the substantia nigra (−2.8 mm to −3.88 mm from the bregma), and tissue staining was performed using GFAP antibody (Neuromics, Edina, MN, USA) in the same manner as in Example 3-2. Intensity was measured using ImageJ software to measure the differences in the diseased mouse control group compared to the normal mouse control group, or the diseased mouse drug administration group compared to the diseased mouse control group.

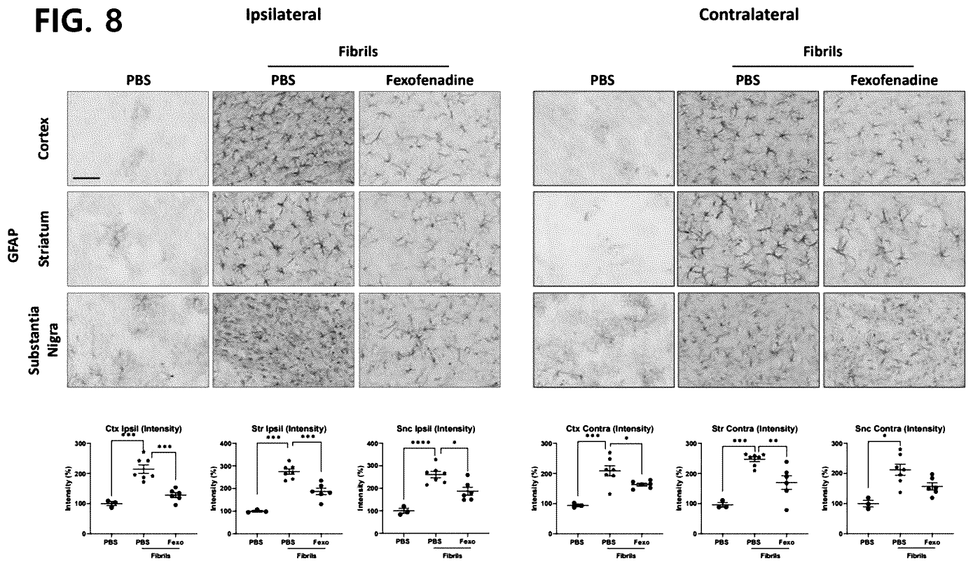

As a result, as shown in FIG. 8, in the diseased mouse control group (PFF-PBS), GFAP expression was significantly increased compared to the normal mouse control group (PBS) in the cortex, striatum and substantia nigra on the ipsilateral and contralateral sides to which α-synuclein was injected. However, in the diseased mouse drug administration group (PFF-fexofenadine), it could be confirmed that GFAP expression was significantly reduced in the cortex, striatum and substantia nigra on the ipsilateral and contralateral sides to which α-synuclein was injected compared to the diseased mouse control group (PFF-PBS).

From the above results, it was confirmed that fexofenadine exerts a protective effect on neurons by suppressing an abnormal increase in the number and activation of astrocytes caused by the destruction of neurons.

### 5-3. Microglial Staining Results in Cortex, Striatum and Substantia Nigra

Using the Parkinson's disease animal model produced in Example 4, the inventors of the present invention attempted to analyze the effect of fexofenadine on microglial activation.

For this purpose, IBA-1 staining was performed using the same method as in Example 3-2, using 6 sheets per mouse from the area including the cortex (from 0.98 mm to −0.10 mm from the bregma), the area including the striatum (from 0.98 nm to −0.10 mm from the bregma), and the area including the substantia nigra (from −2.8 mm to −3.88 mm from the bregma).

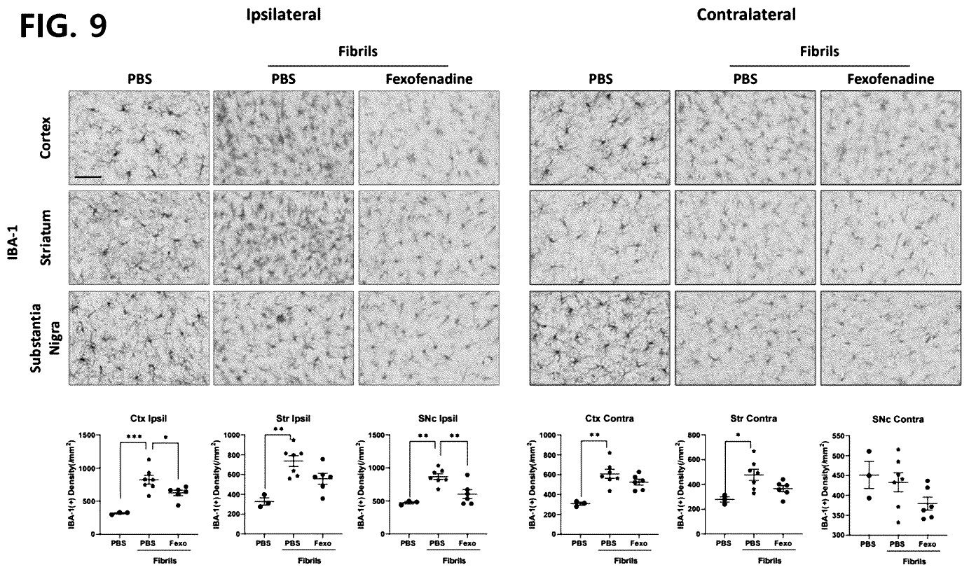

As a result, as shown in FIG. 9, in the diseased mouse control group (PFF-PBS), the density of IBA-1 positive cells was significantly increased in the cortex, striatum and substantia nigra on the ipsilateral side to which α-synuclein was injected, compared to the normal mouse control group (PBS). However, it was confirmed that in the diseased mouse drug-administered group (PFF-fexofenadine), the density of IBA-1 positive cells was significantly reduced in the cortex, striatum and substantia nigra of the ipsilateral side to which α-synuclein was injected compared to the diseased mouse control group (PFF-PBS).

Meanwhile, in the diseased mouse control group (PFF-PBS), the density of IBA-1 positive cells was significantly increased in the cortex and striatum on the contralateral side to which α-synuclein was injected, compared to the normal mouse control group (PBS), but it was confirmed that in the diseased mouse drug administration group (PFF-fexofenadine), the density of IBA-1 positive cells was decreased compared to the diseased mouse control group (PFF-PBS).

From the above results, it can be further proven that fexofenadine has the effect of suppressing inflammation-mediated neuronal damage and neuronal degeneration and protecting neurons by inhibiting the activation of microglial cells against neuronal cytotoxicity.

### Example 6. BIFC-α-Syn C. elegans Verification Using Parkinson's Disease Model

Protein aggregates called Lewy bodies are formed in the neurons of the midbrain substantia nigra of Parkinson's disease patients, and it is known that α-synuclein aggregates in Lewy bodies, thereby causing neurotoxicity.

Meanwhile, BiFC (Bimolecular Fluorescence Complementation) analysis is a method based on the reconstruction of two non-fluorescent fragments derived from a fluorescent protein, and the non-fluorescent protein fragments are divided into an N-terminal Venus (VN) fragment and a C-terminal Venus (VC) fragment, and when A and B, which are linked to VN and VC, respectively, bind, they are reconstituted into a single fluorescent protein, Venus, which causes fluorescence to be expressed. In this case, when α-Synuclein is expressed to be linked to VN and VC, fluorescence is expressed as α-synuclein forms aggregates.

Accordingly, the present invention sought to verify whether fexofenadine inhibits the formation of α-Synuclein aggregates using the BiFC-α-syn C. elegans Parkinson's disease model.

For this purpose, A C. elegans Parkinson's disease model (Venus-αS) expressing BiFC-α-syn was produced by introducing plasmids that overexpress VN-linked α-synuclein in muscle cells of the pharynx and VC-linked α-synuclein in neurons that form synapses with muscle cells into wild-type Bristol N2 C. elegans purchased from the Caenorhabditis Genetics Center (CGC, University of Minnesota, St. Paul, MN) (Kim D-K et al., (2016). Autophagy. 12(10):1849-1863).

L4-stage C. elegans (Venus-αS) models expressing BiFC-α-syn were transferred to plates including DMSO (Dimethyl sulfoxide) or 50 PM fexofenadine drug and cultured in an incubator at 20° C. for 2 days. The control group and the fexofenadine drug-treated C. elegans group were placed in M9 buffer (22 mM KH₂PO₄, 22 mM Na₂HPO₄, 85 mM NaCl, 1 mM MgSO₄) including 10 mM sodium azide, and after being fixed and dispensed into a 96-well plate, the intensity of VENUS fluorescence, was measured using an in cell fluorescence scanning device (Cytiva).

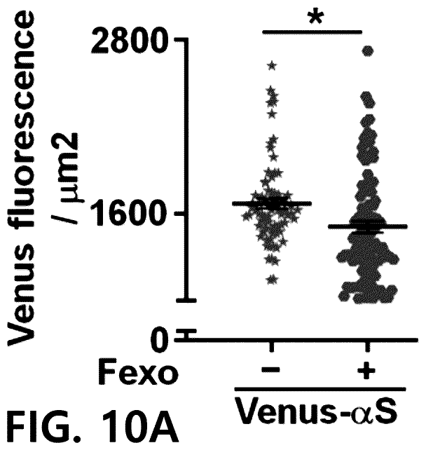

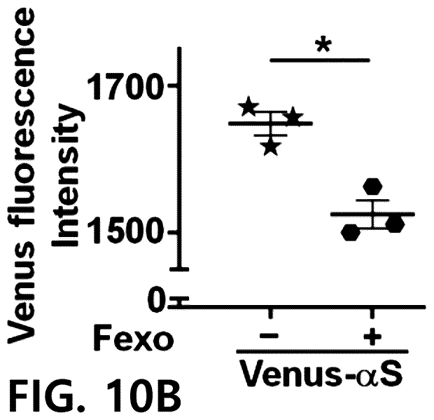

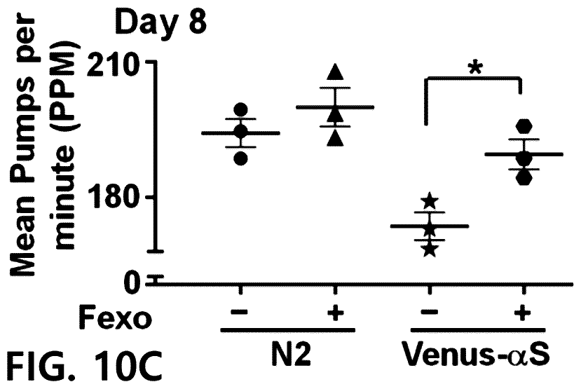

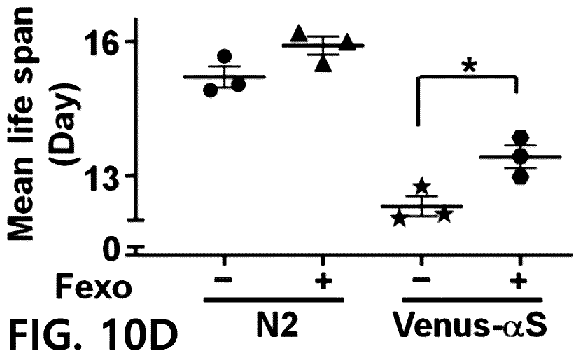

As a result, as shown in FIGS. 10a and 10b, compared with the Venus fluorescence intensity of the control group that was not treated with fexofenadine, the Venus fluorescence intensity was significantly reduced in the C. elegans group that was treated with fexofenadine, and it could be confirmed that fexofenadine inhibits the formation of α-synuclein transaggregates.

Meanwhile, pharyngeal pumping analysis was conducted to confirm whether fexofenadine treatment restores damaged motor skills in the C. elegans Parkinson's disease model. Pharyngeal pumping was counted for 1 minute at room temperature using a light microscope 8 days after fexofenadine treatment, and data were expressed in PPM (pumps per minute).

As a result, as shown in FIG. 10c, the pharyngeal pumping ability of C. elegans was drastically reduced compared to the wild-type N2 in the C. elegans Parkinson's disease model, but When fexofenadine was treated in the C. elegans Parkinson's disease model, it was confirmed that the pharyngeal pumping ability of C. elegans was restored to a level similar to that of wild-type N2. Interestingly, it was confirmed that treatment of C. elegans wild-type N2 with fexofenadine also improves the pharyngeal pumping ability of C. elegans.

Lastly, life-span analysis was conducted to determine the effect of fexofenadine treatment on the lifespan of the C. elegans Parkinson's disease model. For this purpose, eggs laid by adults were grown to the L4 larval stage on NGM plates inoculated with K col. OP50 (CGC) at 20° C. When the L4 stage was reached, they were transferred to NGM plates including 100 mM 5-fluoro-2′-deoxyuridine (Sigma-Aldrich, F0503) to prevent progeny production. The number of living and dead C. elegans was recorded every 1 or 2 days. Worms that burst, were buried or crawled out of the plate were also counted. Survival data were analyzed using OASIS2 (Survival Analysis Online Application for Lifespan Analysis http://sbi.postech.ac.kr/oasis/surv/).

As a result, as shown in FIG. 10d, the lifespan of C. elegans wild-type N2 was about 15 days, whereas the lifespan of the C. elegans Parkinson's disease model was shortened to about 12 days, but the lifespan of the fexofenadine-treated C. elegans Parkinson's disease model was about 14 days, and thus, it was confirmed that fexofenadine extends the lifespan of the C. elegans Parkinson's disease model. Interestingly, it was confirmed that the lifespan of C. elegans was further extended when C. elegans wild-type N2 was treated with fexofenadine.

As the specific parts of the present invention have been described in detail above, it will be clear to those skilled in the art that these specific techniques are merely preferred embodiments and do not limit the scope of the present invention. Accordingly, the actual scope of the present invention will be defined by the appended claims and their equivalents.

### NATIONAL RESEARCH AND DEVELOPMENT PROJECT THAT SUPPORTED THIS INVENTION

[Project Identification Number] 1711161142
[Project Number] 2019R1A5A2026045
[Name of Ministry] Ministry of Science and ICT
[Name of Project Management (Specialized) Institution] National Research Foundation of Korea.
[Title of Research Project] Leading research center support project basic medical science field (MRC)
[Title of Research Task] Brain disease convergence research center
[Contribution Ratio] 1/1
[Name of Task Performance Institution]] Ajou University
[Research Period] Jun. 1, 2019 to Feb. 28, 2026

1. A method for preventing, ameliorating or treating a neurodegenerative disease, comprising administering fexofenadine or a pharmaceutically acceptable salt thereof as an active ingredient to a subject in need thereof.

2. The method of claim 1, wherein the neurodegenerative disease is selected from the group consisting of Parkinson's disease, Alzheimer's disease, Huntington's disease, amyotrophic lateral sclerosis, multiple sclerosis, Creutzfeldt-Jakob disease, Lou Gehrig's disease, spinal amyotrophic lateral sclerosis, Niemann-Pick disease, synucleinopathy and dementia.

3. (canceled)

4. The method of claim 1, wherein the fexofenadine or a pharmaceutically acceptable salt thereof protects neurons from neurotoxicity.

5. The method of claim 4, wherein the fexofenadine or a pharmaceutically acceptable salt thereof protects neurons through:
(i) inhibiting dopaminergic neuron death;
(ii) inhibiting inflammation-mediated neuronal damage or neuronal degeneration;
(iii) inhibiting α-Synuclein Ser-129 phosphorylation aggregate formation; and/or
(iv) inhibiting an abnormal increase in the number and/or activation of astrocytes.

6. The method of claim 1, wherein the fexofenadine or a pharmaceutically acceptable salt thereof:
(i) improves exercise capacity; and/or
(ii) prolongs the lifespan of the subject to which the fexofenadine or a pharmaceutically acceptable salt thereof is administered.

7-10. (canceled)
